(19) United States
(12) Patent Application Publication
(10) Pub. No.: US 2023/0110216 A1
(43) Pub. Date: Apr. 13, 2023

(54) OPHTHALMIC CHLOROPROCAINE GEL HAVING IMPROVED FUNCTIONALITY

(71) Applicant: SINTETICA S.A., Mendrisio (CH)

(72) Inventors: Augusto Mitidieri, Mendrisio (CH); Elisabeta Donati, Mendrisio (CH); Clara Bianchi, Mendrisio (CH)

(21) Appl. No.: 17/911,429

(22) PCT Filed: Mar. 17, 2021
(86) PCT No.: PCT/IB21/52237

§ 371 Date: Sep. 14, 2022

Related U.S. Application Data
(60) Provisional application No. 62/991,611, filed on Mar. 19, 2020.

(51) Int. Cl. A61K 31/245; A61K 9/06; A61K 47/38; A61K 9/00

(52) U.S. Cl. CPC A61K 31/245; A61K 9/06; A61K 47/38; A61K 9/0048

(57) ABSTRACT

Sterile ophthalmic gels of chloroprocaine having improved functionality, pharmacokinetics, and stability, particularly in terms of clarity, and to methods of making and using same.

### FIELD OF INVENTION

The present invention relates to ophthalmic gels of chloroprocaine having improved functionality, pharmacokinetics, and stability, particularly in terms of clarity, and to methods of making and using same.

### BACKGROUND OF INVENTION

Topical anesthetics are marketed without prescription for the relief of various conditions including sunburn, minor burns, insect bites and stings, poison ivy, poison oak, poison sumac, and minor cuts and abrasions. They are also used during minor surgical procedures. Dentists use them to numb oral tissue before injecting a local anesthetic; ophthalmologists use them to numb the surface of the eye when performing minor surgeries and medical procedures; and otolaryngologists use them when performing procedures in the ear canal. Molecules approved as topical anesthetics in the United States and Europe include tetracaine lidocaine, benzocaine, prilocaine, and oxybuprocaine, among others.

What is needed is a topical anesthetic, particularly an ophthalmic gel, having improved functionality, pharmacokinetics, and stability in comparison to prior art formulations. Also needed are methods of manufacturing an ophthalmic gel that provide a viscous sterile formulation, with consistent physical properties, physical appearance, and anesthetic properties.

### SUMMARY OF THE INVENTION

After extensive research and experimentation, the inventors have developed an ophthalmic gel of chloroprocaine, and methods of making and using the gels, that depend on the admixture of a sterile aqueous drug phase and a separate sterile gel matrix. Thus, in a first principal embodiment the invention provides an ophthalmic gel comprising an acidic aqueous solution of chloroprocaine hydrochloride at a pH of 2.4 to 3.2 admixed with an aqueous matrix of hydroxyethyl cellulose having a viscosity greater than 25,000 cP at 25° C. and a pH optionally greater than 6, wherein: (a) the gel comprises 3% chloroprocaine hydrochloride; (b) the gel has a pH of 2.8-3.8; and (c) the matrix viscosity is measured by a BrookField DV III+Pro Spindle 3 at 20 rpm, as described in section 2.2.10 of the European Pharmacopeia 2016 edition.

The gels of the present invention flow in drops that are easy to administer, remain on the eye surface for continued anesthetic effect, and remain clear for ease of surgical procedures. This flow is both non-Newtonian and pseudo-plastic, and is the result of the the unique excipients used in the formulation, and the unique manufacturing process. Thus, in a second principal embodiment the invention provides an ophthalmic gel exhibiting non-Newtonian pseudo-plastic behavior comprising: (a) 3% of chloroprocaine hydrochloride; (b) from 1.0% to 1.25% hydroxyethyl cellulose; (c) hydrochloric acid q.s. to pH 2.8-3.8; and (d) water.

A third principal embodiment relates to the use of any of the formulations of the present invention to induce analgesia in the eye. The methods have been found to be particularly useful when performed in conjunction with cataract surgeries, involving small incisions to the cornea, phacoemulsification, and lens replacement. Thus, in a third principal embodiment the invention provides a method of inducing anesthesia or analgesia on the corneal surface comprising applying to the corneal surface a drop comprising from 0.03 to 0.1 g of the gel of the current invention.

A fourth principal embodiment relates to the method of manufacturing the formulations of the current invention. Thus, in a fourth principal embodiment the invention provides a method of making a 3% chloroprocaine hydrochloride ophthalmic gel comprising: (a) admixing hydroxyethyl cellulose and water to make an aqueous matrix having an initial viscosity greater than 40,000 cP at 25° C. measured by a BrookField DV III+Pro Spindle 3 at 20 rpm, as described in section 2.2.10 of the European Pharmacopeia 2016 edition; (b) thermally sterilizing the aqueous matrix at a temperature of greater than 35 or 40° C. (preferably no more than 60° C.), reducing the viscosity of the aqueous matrix by no more than 40%; (c) admixing chloroprocaine hydrochloride with water and hydrochloric acid to prepare an aqueous acidic solution at a temperature of 35 or 40° C. or greater (preferably no more than 60° C.), having a pH of from 2.4 to 3.2; (d) filter sterilizing the aqueous acidic solution at a temperature of 35 or 40° C. or greater (preferably no more than 60° C.); (e) mixing the aqueous matrix and the aqueous acidic solution to make the gel; and (f) filling the gel into containers.

Additional advantages of the invention are set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 is a schematic of the preferred manufacturing process for the gels of the present invention.

### DETAILED DESCRIPTION

### Definitions and Use of Terms

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

As used in the specification and claims, the singular forms a, an, and the include plural references unless the context clearly dictates otherwise. For example, the term “a pharmaceutical excipient” refers to one or more pharmaceutical excipients for use in the presently disclosed formulations and methods.

When used herein the term “about” will compensate for variability allowed for in the pharmaceutical industry and inherent in pharmaceutical products, such as differences in product strength due to manufacturing variation and time-induced product degradation. In one embodiment the term allows for any variation which in the practice of pharmaceuticals would allow the product being evaluated to be considered pharmaceutically equivalent or bioequivalent to the recited strength. In another embodiment the term allows for any variation within 5% of the recited strength or concentration of the formulation.

The terms “treating” and “treatment,” when used herein, refer to the medical management of a patient with the intent to cure, ameliorate, stabilize, or prevent a disease, pathological condition, injury, or disorder (collectively “disorder”). This term includes active treatment, that is, treatment directed specifically toward the improvement of a disorder, and also includes causal treatment, that is, treatment directed toward removal of the cause of the associated disorder. In addition, this term includes palliative treatment, that is, treatment designed for the relief of symptoms rather than the curing of the disorder; preventative treatment, that is, treatment directed to minimizing or partially or completely inhibiting the development of the disorder; and supportive treatment, that is, treatment employed to supplement another specific therapy directed toward the improvement of the disorder.

As used herein, “therapeutically effective amount” refers to an amount sufficient to elicit the desired biological response. The therapeutically effective amount or dose will depend on the age, sex and weight of the patient, and the current medical condition of the patient. The skilled artisan will be able to determine appropriate dosages depending on these and other factors in addition to the present disclosure.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. “Pharmaceutically acceptable salts” means salts that are pharmaceutically acceptable, as defined above, and which possess the desired pharmacological activity.

When a compound is expressed without indicating whether it is present as a free base or a salt, it will be understood to include both the free base and salt forms. In like manner, when a range of weights, doses, or ratios for a compound is given, it will be understood to refer to the range can be calculated based on the weight of the free base or the salt, unless a particular salt is mentioned, in which case the weight shall refer to the weight of the mentioned salt. Thus, when reference is made to 100 mg chloroprocaine, or 100 mg of chloroprocaine or a pharmaceutically acceptable salt thereof, the disclosure will be understood to encompass 100 mg of chloroprocaine hydrochloride based on the weight of the free base or 100 mg chloroprocaine hydrochloride based on the weight of the salt, among other salts. When reference is made to 100 mg chloroprocaine hydrochloride, the disclosure will be understood only to encompass 100 mg of chloroprocaine hydrochloride based on the weight of the salt.

When ranges are expressed herein by specifying alternative upper and lower limits of the range, it will be understood that the endpoints can be combined in any manner that is mathematically feasible. Thus, for example, a range of from 50 or 80 to 100 or 70 can alternatively be expressed as a series of ranges of from 50 to 100, from 50 to 70, and from 80 to 100. When a series of upper bounds and lower bounds are related using the phase and/or, it will be understood that the upper bounds can be unlimited by the lower bonds or combined with the lower bounds, and vice versa. Thus, for example, a range of greater than 40% and/or less than 80% includes ranges of greater than 40%, less than 80%, and greater than 40% but less than 80%.

When percentages, concentrations or other units of measure are given herein, it will be understood that the units of measure are weight percent unless otherwise stated to the contrary.

The weight average molecular weight (Mₙ) is defined by the following formula:

$$Mn = \frac{\sum {N}_{i} {M}_{i}}{\sum {N}_{i}}$$

where Mᵢ is the molecular weight of a chain and Nᵢ is the number of chains of that molecular weight. Compared to number weight average molecular weight, weight average molecular weight takes into account the molecular weight of a chain in determining contributions to the molecular weight average. The more massive the chain, the more the chain contributes to Mₙ. Mₙ is determined by methods that are sensitive to the molecular size rather than just their number, such as light scattering techniques.

When the stability of a drug product over time is given herein, it will be understood to be evaluated by standard methods known to those skilled in the art, as prescribed in ICH Q1A(R2) Stability Testing of New Drug Substances and Products (November 2003), typically evaluated and reported using HPLC.

“Total impurities” refers to by-products formed during the synthesis of chloroprocaine hydrochloride, residual impurities remaining from the synthesis of chloroprocaine hydrochloride after purification, and impurities caused by the degradation of chloroprocaine during manufacture or storage, as further defined and evaluated under ICH Q3A(R2) Impurities in New Drug Substances (October 2006), typically evaluated nd reported using HPLC.

### Discussion of Principal Embodiments

The invention can be defined based on several principal embodiments which can be combined in any manner physically and mathematically possible to create additional principal embodiments.

A first principal embodiment provides an ophthalmic gel comprising an acidic aqueous solution of chloroprocaine hydrochloride at a pH of 2.4 to 3.2 admixed with an aqueous matrix of hydroxyethyl cellulose having a viscosity greater than 25,000 cP at 25° C. and a pH optionally greater than 6, wherein: (a) the gel comprises 3% chloroprocaine hydrochloride; (b) the gel has a pH of 2.8-3.8; and (c) the matrix viscosity is measured by a BrookField DV III+Pro Spindle 3 at 20 rpm, as described in section 2.2.10 of the European Pharmacopeia 2016 edition.

A second principal embodiment provides an ophthalmic gel exhibiting non-Newtonian pseudo-plastic behavior comprising: (a) 3% of chloroprocaine hydrochloride; (b) from 1.0% to 1.25% hydroxyethyl cellulose; (c) hydrochloric acid q.s. to pH 2.8-3.8; and (d) water.

A third principal embodiment provides a method of inducing anesthesia or analgesia on the corneal surface comprising applying to the corneal surface a drop comprising from 0.03 to 0.1 g of the gel of the current invention.

A fourth principal embodiment provides a method of making a 3% chloroprocaine hydrochloride ophthalmic gel comprising: (a) admixing hydroxyethyl cellulose and water to make an aqueous matrix having an initial viscosity greater than 40,000 cP at 25° C. measured by a BrookField DV III+Pro Spindle 3 at 20 rpm, as described in section 2.2.10 of the European Pharmacopeia 2016 edition; (b) thermally sterilizing the aqueous matrix at a temperature of greater than 35 or 40° C. (preferably no more than 60° C.), reducing the viscosity of the aqueous matrix by no more than 40%; (c) admixing chloroprocaine hydrochloride with water and hydrochloric acid to prepare an aqueous acidic solution at a temperature of 35 or 40° C. or greater (preferably no more than 60° C.), having a pH of from 2.4 to 3.2; (d) filter sterilizing the aqueous acidic solution at a temperature of 35 or 40° C. or greater (preferably no more than 60° C.); (e) mixing the aqueous matrix and the aqueous acidic solution to make the gel; and (f) filling the gel into containers.

The invention can further be understood with reference to various subembodiments which can modify any of the principal embodiments. These subembodiments can be combined in any manner that is both mathematically and physically possible to create additional subembodiments, which in turn can modify any of the principal embodiments.

### Discussion of Formulation Subembodiments

In some embodiments, the gel is characterized by the concentration of hydroxyethylcellulose in the final formulation. Thus, in some embodiments the gel comprises from 1.0% to 1.25% hydroxyethylcellulose. In other embodiments, the gel comprises from 1.04% to 1.14% hydroxyethyl cellulose. In still further ambodiments the gel comprises a hydroxyethyl cellulose concentration of about 1.09%, most preferably at a pH of 3.0-3.4.

The gel can also be characterized by its pH and in any of the embodiments of the present invention the gel has a pH of from 2.5 to 4.5, from 2.6 to 4.0, from 2.8 to 3.8, or from 3.0 to 3.4.

In other embodiments, the gel is characterized by the hydroxyethylcellulose used in the gel. In some embodiments the hydroxyethyl cellulose has a weight average molecular weight of from 800,000 to 2,000,000 daltons, from 1,000,000 daltons to 1,500,000 daltons, from 1,250,000 to 1,350,000 daltons, or about 1,300,000 daltons. In any of the embodiments of the present invention, the hydroxyethyl cellulose preferably exhibits non-Newtonian pseudo-plastic behavior.

The gel also can be characterized by its viscosity, when measured by a BrookField DV II+Pro Spindle 3 at 100 rpm, as described in section 2.2.10 of the European Pharmacopeia 2016 edition. Thus, in some embodiments the gel has a viscosity of from 1000 to 2000 cP at 25° C. In further embodiments the gel has a viscosity of from 1000 to 1500 cP at 25° C. In another embodiment the gel has a viscosity of from 1500 to 2000 cP at 25° C.

The gel also can be characterized by the remarkably low levels of ACBA (4-amino-2-chlorobenzoic acid) in the finished formulation, and in various embodiments the gel comprises less than 3.0% ACBA, less than 1.0% ACBA, less than 0.4% ACBA, less than 0.2% ABCA, less than. 0.1% ABCA, or even less than 0.05% ACBA. These figures apply at the completion of manufacturing the product as well and throughout the product's shelf life, including after two years of storage protected from light at 25° C. and 40% relative humidity.

The gel also can be characterized by its remarkably low levels of total impurities (as defined herein), at the completion of manufacturing the product as well as throughout the product's shelf life. Thus, for example, in any of the embodiments described herein, the gel preferably compriuses less than 0.6% or 0.4% total impurities after a period of six months of storage protected from light at 20° C. and 40% relative humidity. At any time point after manufacture, the gel preferably comprises less than 0.4% total impurities.

### Methods of Treatment

The formulations have been found effective for inducing local anesthesia or analgesia on the corneal surface, and can be used during ocular surgery or in response to a corneal abrasion or trauma. Particularly suitable surgeries for practicing the present invention include, for example, cataract surgery, treatment for maculopathy, conventional glaucoma surgery, vitrectomy, surgeries for diabetic nephropathy, and various laser surgeries including laser-assisted in situ keratomileusis and photorefractive keratectomy. The formulations induce local analgesia or anesthesia in the eye, and they do so without inducing significant irritation.

A preferred surgical procedure is phacoemulsification for removal of a senile or pre-senile cataract which includes an incision through the cornea, capsulorhexis, phacoemulsification, and intra-ocular lens implantation.

A preferred dosing regimen is as follows:

1st Drop instillation, then wait for about 5 minutes

Eye Disinfection, then wait for about 2 minutes

2nd Drop instillation, then wait for about 1 minute

3^{rd} Drop instillation, then wait for about 1 minute

Start of Surgery.

Two other preferred dosing regimens are as follows:
3 drops (1 instillation every minute±15 seconds)
3+3 drops (1 instillation every minute±15 seconds for the first 3 drops, then wait for about 5 minutes before new instillation every minute 35 15 seconds for the last 3 drops)

Alternatively, drops can be instilled at the physician's discretion, either before or during the surgery. The safety of the product supports administering from one to ten drops, at any timepoint before or during surgery.

A preferred drop size ranges from from 0.03 to 0.1 g, from 0.03 to 0.08 g, or from 0.045 to 0.065 g, and preferably induces pharmaceutically effective concentrations of chloroprocaine in the aqueous humor, cornea, and conjuctivae, within at least 10 minutes of installing the first drop, and lasting through at least 30, 45, or 60 minutes after installing the last drop.

### Methods of Manufacture

As noted in the principal embodiments, the gels of the current invention are in one embodiment made by (a) admixing hydroxyethyl cellulose and water to make an aqueous matrix having an initial viscosity greater than 40,000 cP at 25° C. measured by a BrookField DV III+Pro Spindle 3 at 20 rpm, as described in section 2.2.10 of the European Pharmacopeia 2016 edition; (b) thermally sterilizing the aqueous matrix at a temperature of greater than 35 or 40° C., reducing the viscosity of the aqueous matrix by no more than 40%; (c) admixing chloroprocaine hydrochloride with water and hydrochloric acid to prepare an aqueous acidic solution at a temperature of 35 or 40° C. or greater (preferably no more than 60° C.), having a pH of from 2.4 to 3.2; (d) filter sterilizing the aqueous acidic solution at a temperature of 35 or 40° C. or greater (preferably no more than 60° C.); (e) mixing the aqueous matrix and the aqueous acidic solution to make the gel; and (f) filling the gel into containers. The In another embodiment the gel is made by admixing an acidic aqueous solution of chloroprocaine hydrochloride at a pH of 2.4 to 3.2 with an aqueous matrix of hydroxyethyl cellulose having a viscosity greater than 25,000 cP at 25° C. and a pH optionally greater than 6. In further embodiments of the invention, the foregoing manufacturing parameters can be varied as follows:
The initial viscosity of the matrix can exceed 20,000, 30,000, 40,000, or 50,000 cP at 25° C., but preferably does not exceed 100,000 or 60,000 cP at 25° C., as measured by a BrookField DV III+Pro Spindle 3 at 20 rpm, as described in section 2.2.10 of the European Pharmacopeia 2016 edition;
The aqueous matrix can be thermally sterilized at a temperature of greater than 35, 40, 45 or 50° C., preferably ranging from 35 to 45° C. (preferably no more than 60° C.), and most preferably at about 40° C.;
Thermal sterilization of the aqueous matrix preferably reduces its viscosity by no more than 40%, 35%, or 30%, preferably ranging from 5% to 40% or from 10% to 30%;
The viscosity of the aqueous matrix after thermal sterilization preferably exceeds 15,000, 20,000, 25,000, or 30,000 cP at 25° C., but preferably does not exceed 60,000 or 40,000 cP at 25° C. after the thermally sterilizing, as measured by a BrookField DV III+Pro Spindle 3 at 20 rpm, as described in section 2.2.10 of the European Pharmacopeia 2016 edition;
The pH of the aqueous matrix is preferably alkaline (i.e. 7-8), but can also range from 5 to 9, from 6 to 8, or from 6.5 to 7.5.
The aqueous acidic solution can be prepared at a temperature of greater than 35, 40, 45 or 50° C. (preferably no more than 60° C.), preferably ranging from 35 to 45° C., and most preferably at about 40° C.;
The aqueous acidic solution can be prepared, by the addition of HCl, at a pH of from 2.2 to 4.0, from 2.4 to 3.5, from 2.4 to 3.2, from 2.5 to 3.0, or from 2.6 to 2.8.
The aqueous acidic solution can be filter sterilized at a temperature of greater than 35, 40, 45 or 50° C. (preferably no more than 60° C.), preferably ranging from 35 to 45° C., and most preferably at about 40° C., and preferably through a 0.22 micron filter;
The weight ratio of the aqueous acidic solution to the gel matrix preferably ranges from 30:70 to 70:30.

Still further embodiments of the present invention relate to the sequence of the steps in the methods of manufacture of the present invention, and certain negative provisos. Thus, in other embodiment, in the methods of manufacture of the present invention, one, two, or all three of the following additional conditions are met: (i) steps (a) and (b) are performed before steps (c) and (d), (ii) the pH of the formulation is not adjusted after step (e), and/or (iii) the viscosity of the formulation is not adjusted after step (e).

Still further embodiments relate to the containers used to package the gel. Thus, in some embodiments the containers are monodose containers comprising from 0.5 to 2 grams of gel formulation. In other embodiments the containers are multi-dose containers comprising from 1 to 25 grams of gel formulation.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1. Manufacture of Formulation

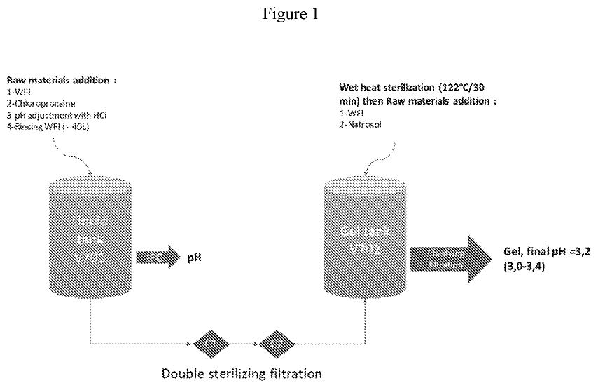

A 3% chloroprocaine gel having the quali-quantitative formulation in Table 1 (CHLO-1708-L02) is manufactured according to the schematic given in FIG. 1, with the following added detail:
the API phase was held at approximately 40° C., and sterile filtration of the API phase was conducted at a temperature of approximately 40° C.;
the API phase contained approximately 0.06-0.07 g/g chloroprocaine HCl;
the API phase was filter sterilized by passing the solution through a 0.22 micron filter;
1N HCl was added to the API phase to obtain a pH of 3.04;
the gel (matrix) phase was thermally sterilized at approximately 40° C.;
the gel (matrix) phase had an initial viscosity of 48,000, which dropped to 33,920 (−29.3%) during thermal sterilization;
the gel (matrix) phase had an initial pH of 7.31 which dropped to 7.06 during sterilization;
the API phase was prepared after the gel phase;
there was no pH modification, by addition of an alkaline or acidic agent, to the formulation after the API and gel phases were combined; and
the resulting product exhibited non-Newtonian behavior and a high degree of pseudo-plasticity.

Viscosity measurements were made of the formulation both during manufacture and after the formulation was finished. All viscosity measurements on the gel/matrix phase were made using a Brookfield DV III Viscosimeter or equivalent, at a water bath temperature of 25±0.5° C. for 15 minutes, at a speed of 20 rpm and run time of 2 minutes. All viscosity measurements on the finished gel were made using a Brookfield DV II Viscosimeter, at a water bath temperature of 25±1° C. for 15 minutes, at a speed of 100 rpm and a run time of 5 minutes.

TABLE 1
Quali-Quantitative Formulation

| Ingredient | Quantity (% w/v) |
|---|---|
| Chloroprocaine HCl | 3.0%* |
| Hydroxy ethyl Cellulose** | 1.09% |
| 1N Hydrochloric Acid | q.s. to pH 3.0-3.4 |
| WFI | q.s. to 100% |

*Calculated based on the weight of the anhydrous hydrochloride salt.
**The hydroxyethyl cellulose is characterized by a weight average molecular weight (Da) of 1,300,000, a NF Brookfield LVF viscosity at 25° C., (mPa · s in 1% solution) of 3.500-5,500, and an EP viscosity at 10 s⁻¹ and 25° C. (mPa · s in 2% solution) of 14,500.

The finished product had a viscosity of approximately 1247-1260 cP, an osmolality between 152 and 158 mOsmol/kg, and an ACBA impurity content of 0.04-0.05%.

### Example 2. Stability Studies

Further studies were undertaken to determine the stability of the API phase at various temperatures and pH adjustments, as reported in Tables 2a, 2b, and 2c, based on the generation of ACBA impurities in solution.

TABLE 2a

| | | | Temperature |
|---|---|---|---|
| | Temperature of | Adjustment | of the solution |
| | API Dissolution | of the pH | during the study |
| Solution # 1 | 40° C. | No adjustment | 25° C. |
| | | (pH = 4.58) | |
| Solution # 2 | 40° C. | Adjustment to | 25° C. |
| | | pH = 3.0 | |
| Solution # 3 | 40° C. | No adjustment | 40° C. |
| | | (pH = 4.58) | |
| Solution # 4 | 40° C. | Adjustment to | 40° C. |
| | | pH = 3.0 | |

TABLE 2b

| | Appearance | |
|---|---|---|
| Time (h) | 0 | 48 |
| Solution # 1 | Clear solution, free from | Turbid solution, free from |
| | visible particles | visible particles |
| Solution # 2 | Clear solution, free from | Turbid solution, containing |
| | visible particles | visible particles |
| Solution # 3 | Clear solution, free from | Clear solution, free from |
| | visible particles | visible particles |
| Solution # 4 | Clear solution, free from | Clear solution, free from |
| | visible particles | visible particles |

TABLE 2c

| | ACBA (4-Amino-2-chlorobenzoic acid) Assay (%) | | |
|---|---|---|---|
| Time (h) | 0 | 24 | 48 |
| Solution # 1 | 0.004 | 0.019 | 0.028 |
| Solution # 2 | 0.004 | 0.005 | 0.006 |
| Solution # 3 | 0.010 | 0.066 | 0.093 |
| Solution # 4 | 0.005 | 0.013 | 0.020 |

As can be seen, a clearer solution was obtained when the temperature of the API phase was maintained at 40° C., and the pH of the API phase was adjusted to approximately 3.0, prior to combination with the gel/matrix phase.

### Example 3. Stability Study of Finished Formulation

The formulations described in Example 1 was tested for stability after six months of storage at 25° C. and 40% Relative Humidity protected from light. Methods for performing the stability analyses are described in Table 3a. The results of the stability testing are reported in Table 3b.

TABLE 3a
Test Items and Analytical Procedures

| Test Item | Analytical Procedure |
|---|---|
| Appearance | visual |
| Viscosity (mPas) | BrookField DV II + Pro |
| | Spindle, 100 rpm |
| | Ref. Ph Eur 2.2.10 current edition |
| Chloroprocaine HCl Assay | HPLC |
| Impurity ACBA % | HPLC |
| Impurity DAHB Assay | HPLC |
| Unknown impurity | HPLC |

TABLE 3b
Stability of Example 1 Formulation

| | T0 | T3 | T6 | T9 | T12 | T18 | T24 |
|---|---|---|---|---|---|---|---|
| Appearance | passes | passes | passes | passes | passes | passes | passes |
| Viscosity | 1252 | 1178 | 1185 | 1200 | 1138 | 1075 | 1023 |
| Chloroprocaine Assay | 100.1 | 100.3 | 98.6 | 99.3 | 99.9 | 99.1 | 100.5 |
| ACBA Assay | 0.05 | 0.31 | 0.52 | 0.70 | 0.91 | 1.2 | 1.6 |
| DAHB Assay | <0.05 | <0.05 | <0.05 | <0.05 | <0.05 | <0.05 | <0.05 |
| Total Unknown Impurities | <0.05 | <0.05 | <0.05 | <0.05 | <0.05 | <0.05 | <0.05 |

### Example 4. Pharmacokinetic Testing of Example 1 Formulation; Single Dose Administration

A study was conducted by instilling a 50 μl volume of the gel described in Example 1 into the right eye of 42 white albino rabbits (2-2.5 kg), visually inspecting for irritation or toxicity through an ophthalmoscope, euthanizing the animal, extracting samples from the aqueous humor, cornea, and conjunctivae, and analyzing the samples for chloroprocaine content. When necessary, tissue concentrations were calculated by extrapolating the test results outside the validated concentration ranges, as reported in Table 4.

TABLE 4
Chloroprocaine content in ocular tissues
Mean concentrations of chloroprocaine (ng/mL) in ocular tissues
at different time points (chloroprocaine 3% gel)

| Time point (min) | Aqueous Humor | Cornea | Conjuctivae |
|---|---|---|---|
| 10 | 16,435 | 433,758 | 420,931 |
| 20 | 12,230 | 244,704 | 150,181 |
| 30 | 11,433 | 205,833 | 151,096 |
| 45 | 8,625 | 108,165 | 46,858 |
| 60 | 9,139 | 150,256 | 44,716 |
| 75 | 4,534 | 54,832 | 7,589 |
| 90 | 1,119 | 14,658 | 4,833 |

The drug was well tolerated based on the Draize test, which evaluates ocular irritation under an ophthalmoscope. Slight conjunctival redness (score 1/3) was observed immediately before euthanasia for the following rabbits: Rabbits 5 and 6 treated eyes (10 min post dose), Rabbit 9 left untreated eye (20 min post dose), Rabbit 15 treated eye (30 min post dose), Rabbit 24 treated eye (45 min post dose), Rabbit 30 treated and untreated eye (60 min post dose), Rabbit 37 treated eye (90 min post dose).

### Example 5. Pharmacokinetic Testing of Example 1 Formulation; Two Dose Administration

The aim of this study was to obtain data on the PK profile of chloroprocaine in aqueous humor, cornea and conjunctivae (both bulbar & palpebral) ocular tissues after two instillations of the formulation of Example 1, with a 10 min-interval, of chloroprocaine eyedrops gel in the right eyes of albino rabbits followed by RRLC-MS/MS analysis. The results are presented in Table 5.

TABLE 5
Chloroprocaine content in ocular tissues

| | Aqueous humor (ng/mL) | | | | | Conjunctivae (ng/g) | | |
|---|---|---|---|---|---|---|---|---|
| | Concentration of | | | | | Concentration of | | |
| | chloroprocaine in | | | % of | | chloroprocaine in | | |
| Time-point | ocular tissues | | | applied dose | | ocular tissues | | |
| (hours) | mean | sd | n | mean | sd | mean | sd | n |
| 0.17 | 19 069 | 6 095 | 6 | 1.2E−01 | 4.2E−02 | 130 785 | 135 927 | 5 |
| 0.75 | 7 664 | 13 060 | 6 | 4.7E−02 | 7.7E−02 | 38 926 | 68 111 | 6 |
| 1.5 | 63 | 54 | 6 | 4.3E−04 | 3.6E−04 | 4 136 | 2 922 | 6 |
| 4 | 11 | 26 | 6 | 7.6E−05 | 1.9E−04 | 3 839 | 1 251 | 6 |
| 8 | 103 | 156 | 6 | 6.4E−04 | 9.3E−04 | 2 786 | 2 430 | 5 |
| 24 | 11 | 27 | 6 | 8.9E−05 | 2.2E−04 | 5 466 | 5 224 | 6 |
| 48 | 6 | 15 | 6 | 3.6E−05 | 8.7E−05 | 1 913 | 3 194 | 6 |

PK Parameters

| | | |
|---|---|---|
| Cₘₐₓ ± SD | 19 069 ± 6 095 | 130 785 ± 135 927 |
| Tₘₐₓ | 0.17 | 0.17 |
| t_{1/2} (hours) | 10.4 | ND |
| AUC_{0.17-48 hours} | 12 136 | 243 433 |

| | | | Cornea (ng/g) | | | | |
|---|---|---|---|---|---|---|---|
| | Conjunctivae (ng/g) | | Concentration of | | | | |
| | % of | | chloroprocaine in | | | % of | |
| Time-point | applied dose | | ocular tissues | | | applied dose | |
| (hours) | mean | sd | mean | sd | n | mean | sd |
| 0.17 | 5.8E−01 | 5.5E−01 | 359 916 | 226 863 | 6 | 6.8E−01 | 3.9E−01 |
| 0.75 | 1.8E−01 | 3.2E−01 | 101 767 | 195 420 | 6 | 2.1E−01 | 4.1E−01 |
| 1.5 | 1.9E−02 | 1.5E−02 | 6 813 | 6 382 | 6 | 1.3E−02 | 1.3E−02 |
| 4 | 1.5E−02 | 5.1E−03 | 3 637 | 3 567 | 6 | 7.7E−03 | 8.0E−03 |
| 8 | 1.1E−02 | 8.9E−03 | 13 392 | 20 214 | 6 | 2.3E−02 | 3.1E−02 |
| 24 | 2.5E−02 | 2.5E−02 | 1 377 | 3 029 | 6 | 2.6E−03 | 5.7E−03 |
| 48 | 8.0E−03 | 1.2E−02 | 636 | 985 | 6 | 1.2E−03 | 1.8E−03 |

| PK Parameters | | |
|---|---|---|
| Cₘₐₓ ± SD | 130 785 ± 135 927 | 359 916 ± 226 863 |
| Tₘₐₓ | 0.17 | 0.17 |
| t_{1/2} (hours) | ND | 9.5 |
| AUC_{0.17-48 hours} | 243 433 | 364 814 |

sd = standard deviation
n = number of the eyes
Cₘₐₓ = the highest mean value measured (ng/g or mL of tissue)
Tₘₐₓ = time-point when the highest mean value is measured (hours)
t½ = ln(2)/(−a) with a = slope of ln (concentration) = f(t)
t½ calculated in the decrease phase with minimum 3 values without Cmax, and r2 >0.8
AUC time 1 to time 2 = area under the curve in ng/g or mL of tissue
ND = not determined, r2 of regression in decrease phase <0.8 (0.74)
Applied dose = 3 000 000 ng/eye

No abnormal behavior or unhealthy signs were found for any animal during the in-vivo phase.

### OTHER EMBODIMENTS

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An ophthalmic gel comprising an acidic aqueous solution of chloroprocaine hydrochloride at a pH of 2.4 to 3.2 admixed with an aqueous matrix of hydroxyethyl cellulose having a viscosity greater than 25,000 cP at 25° C. and a pH optionally greater than 6, wherein:
a) the gel comprises 3% chloroprocaine hydrochloride;
b) the gel has a pH of 2.8-3.8; and
c) the matrix viscosity is measured by a BrookField DV III+Pro Spindle 3 at 20 rpm, as described in section 2.2.10 of the European Pharmacopeia 2016 edition.

2. An ophthalmic gel exhibiting non-Newtonian pseudo-plastic behavior comprising:
a) 3% of chloroprocaine hydrochloride;
b) from 1.0% to 1.25% hydroxyethyl cellulose;
c) hydrochloric acid q.s. to pH 2.8-3.8; and
d) water.

3. The gel of claim 1 wherein the acidic aqueous solution of chloroprocaine hydrochloride is filter sterilized and the aqueous matrix of hydroxyethyl cellulose is thermally sterilized.

4. The gel of claim 1 wherein the acidic aqueous solution of chloroprocaine hydrochloride is filter sterilized at a temperature greater than 35 or 40° C. and the aqueous matrix of hydroxyethyl cellulose is thermally sterilized at a temperature greater than 35 or 40° C.

5. The gel of claim 1, 3, or 4 wherein the aqueous matrix of hydroxyethyl cellulose has a pH of from 6 to 8.

6. The gel of claim 1 or 3-5 wherein the aqueous matrix of hydroxyethyl cellulose loses less than 40% of its viscosity when subjected to thermal sterilization.

7. The gel of claim 1 or 3-5 wherein the aqueous matrix of hydroxyethyl cellulose loses from 10 to 30% of its viscosity when subjected to thermal sterilization.

8. The gel of any of the foregoing claims comprising from 1.04% to 1.14% hydroxyethyl cellulose.

9. The gel of any of the foregoing claims having a hydroxyethyl cellulose concentration of about 1.09% and a pH of 3.0-3.4.

10. The gel of any of the foregoing claims wherein the hydroxyethyl cellulose has a weight average molecular weight of from 1,000,000 daltons to 1,500,000 daltons.

11. The gel of any of the foregoing claims wherein the gel has a viscosity of from 1000 to 2000 cP at 25° C. when measured by a BrookField DV II+Pro Spindle 3 at 100 rpm, as described in section 2.2.10 of the European Pharmacopeia 2016 edition.

12. The gel of any of the foregoing claims wherein the gel has a viscosity of from 1000 to 1500 cP at 25° C. when measured by a BrookField DV II+Pro Spindle 3 at 100 rpm, as described in section 2.2.10 of the European Pharmacopeia 2016 edition.

13. The gel of any of the foregoing claims, wherein the hydroxyethyl cellulose exhibits non-Newtonian pseudo-plastic behavior.

14. The gel of any of the foregoing claims in a 0.03-0.1 g drop.

15. The gel of any of the foregoing claims in a 0.045-0.065 g drop.

16. The gel of any of the foregoing claims comprising less than 3.0%, 1.0%, 0.6%, or 0.4% ACBA after a period of 2 years storage protected from light at 25° C. and 40% relative humidity.

17. The gel of any of the foregoing claims comprising less than 0.6% or 0.4% total impurities after a period of six storage protected from light at 20° C. and 40% relative humidity.

18. The gel of any of the foregoing claims comprising less than 0.2% or 0.4% total impurities.

19. A method of manufacturing a 3% chloroprocaine hydrochloride ophthalmic gel comprising:
a) admixing hydroxyethyl cellulose and water to make an aqueous matrix having an initial viscosity greater than 40,000 cP at 25° C. measured by a BrookField DV III+Pro Spindle 3 at 20 rpm, as described in section 2.2.10 of the European Pharmacopeia 2016 edition;
b) thermally sterilizing the aqueous matrix at a temperature of greater than 35 or 40° C., reducing the viscosity of the aqueous matrix by no more than 40%;
c) admixing chloroprocaine hydrochloride with water and hydrochloric acid to prepare an aqueous acidic solution at a temperature of 35 or 40° C. or greater, having a pH of from 2.4 to 3.2;
d) filter sterilizing the aqueous acidic solution at a temperature of 35 or 40° C. or greater;
e) mixing the aqueous matrix and the aqueous acidic solution to make the gel; and
f) filling the gel into containers.

20. The method of claim 19 wherein (i) steps (a) and (b) are performed before steps (c) and (d), (ii) the pH of the formulation is not adjusted after step (e), and (iii) the viscosity of the formulation is not adjusted after step (e).

21. The method of claim 19 or 20, wherein the aqueous matrix has a viscosity of greater than 25,000 cP at 25° C. after the thermally sterilizing, as measured by a BrookField DV III+Pro Spindle 3 at 20 rpm, as described in section 2.2.10 of the European Pharmacopeia 2016 edition.

22. The method of any of claims 19-21 wherein the aqueous matrix of hydroxyethyl cellulose has a pH of from 6 to 8.

23. The method of any of claims 19-22 wherein the aqueous matrix of hydroxyethyl cellulose loses from 10 to 30% of its viscosity when subjected to thermal sterilization.

24. The method of any of claims 19-23 wherein the gel comprises from 1.0% to 1.25% hydroxyethylcellulose and hydrochloric acid q.s. to pH 2.8-3.8.

25. The method of any of claims 19-24 wherein the gel comprises from 1.04% to 1.14% hydroxyethyl cellulose.

26. The method of any of claims 19-25 wherein the gel comprises a hydroxyethyl cellulose concentration of about 1.09% and a pH of 3.0-3.4.

27. The method of any of claims 19-26 wherein the hydroxyethyl cellulose has a weight average molecular weight of from 1,000,000 daltons to 1,500,000 daltons.

28. The method of any of claims 19-27 wherein the gel has a viscosity of from 1000 to 2000 cP at 25° C. when measured by a BrookField DV II+Pro Spindle 3 at 100 rpm, as described in section 2.2.10 of the European Pharmacopeia 2016 edition.

29. The method of any of claims 19-28 wherein the gel has a viscosity of from 1000 to 1500 cP at 25° C. when measured by a BrookField DV II+Pro Spindle 3 at 100 rpm, as described in section 2.2.10 of the European Pharmacopeia 2016 edition.

30. The method of any of claims 19-29 wherein the hydroxyethyl cellulose exhibits non-Newtonian pseudo-plastic behavior.

31. The method of any of claims 19-30 wherein the gel comprises less than 3.0%, 1.0%, 0.6%, or 0.4% ACBA after two years of storage protected from light at 25° C. and 40% relative humidity.

32. The method of any of claims 19-31 wherein the gel comprises less than 0.6% or 0.4% total impurities after a period of six storage protected from light at 20° C. and 40% relative humidity.

33. The method of any of claims 19-32 wherein the gel comprises less than 0.2% or 0.4% total impurities.

34. The method of any of claims 19-33 wherein the containers are monodose containers comprising from 0.5 to 2 grams of gel formulation or multi-dose containers comprising from 1 to 25 grams of gel formulation.

35. A gel manufactured by the method of any of claims 19-34.

36. The gel of claim 35 comprising less than 0.2% or 0.4% total impurities.

37. A method of inducing anesthesia or analgesia on the corneal surface comprising a step 1 of applying to the corneal surface a drop comprising from 0.03 to 0.1 g of the gel of any of claim 1-18 or 35.

38. The method of claim 37 further comprising a step 2 of applying to the corneal surface second and third drops comprising from 0.03 to 0.1 g of the gel approximately one minute apart, and approximately 1-5 minutes after the first step.

39. The method of claim 38 comprising repeating steps 1 and 2 approximately 5 minutes after performing step 2 the first time.

40. The method of claim 37 wherein the drop comprises from 0.045 to 0.065 g of the gel.

41. The method of claim 38 further comprising a step 2 of applying to the corneal surface second and third drops comprising from 0.045 to 0.065 g of the gel approximately one minute apart, approximately 1-5 minutes after the first step.

42. The method of claim 39 comprising repeating steps 1 and 2 approximately 5 minutes after performing step 2 the first time.

43. The method of any of claims 37-42 wherein the method is performed before a surgical procedure on the eye involving a surgical incision of the cornea.
